(19) United States
(12) Patent Application Publication
(10) Pub. No.: US 2021/0000882 A1
(43) Pub. Date: Jan. 7, 2021

(54) PRODUCTION AND USE OF EXTRACELLULAR VESICLES

(71) Applicant: Crown Scientific, LLC, San Antonio, TX (US)

(72) Inventor: Ramon Coronado, San Antonio, TX (US)

(21) Appl. No.: 16/976,435

(22) PCT Filed: Feb. 28, 2019
(86) PCT No.: PCT/US2019/020064

§ 371 Date: Aug. 27, 2020

Related U.S. Application Data
(60) Provisional application No. 62/636,698, filed on Feb. 28, 2018.

(51) Int. Cl. A61K 35/50; A61K 38/21; A61K 31/713; A61P 35/04; A61P 37/06; A61K 35/51

(52) U.S. Cl. CPC A61K 35/50; A61K 38/217; A61K 35/51; A61P 35/04; A61P 37/06; A61K 31/713

(57) ABSTRACT

The invention provides a method of producing and using extracellular vesicles (ECVs) derived from activated stromal cells for the treatment of certain disease and conditions. Specifically, ECVs derived from preactivated mesenchymal cells are effective in reducing cancer cell growth and metastasis as well as inducing tolerogenesis in immature dendritic cells. Additionally, ECVs derived from umbilical cord blood may be useful for immunosuppression and reduction of inflammation.

### CROSS-REFERENCE TO RELATED APPLICATIONS

This application claims the benefit of priority under 35 U.S.C. § 119(e) of U.S. Application Ser. No. 62/636,698 filed Feb. 28, 2018. The disclosure of the prior application is considered part of and is incorporated by reference in the disclosure of this application in its entirety.

### FIELD OF THE INVENTION

The present invention relates generally to the production of extracellular vesicles and more specifically to the use of extracellular vesicle compositions to treat diseases.

### BACKGROUND INFORMATION

Exosomes are a family of small nanoparticles having a diameter comprised between 30 and 100 nm. Exosomes are generated inside multivesicular endosomes or multivesicular bodies (MVBs) and are secreted when these compartments fuse with plasma membrane. Exosome secretion is a general cellular function that plays an important role in the intercellular transfer of information, as such exosomes exert their function through paracrine effects. These secreted vesicles serve as cell-to-cell messengers that modify cellular function in normal state as well as in disease state. These secreted vesicles serve as cell-to-cell messengers that modify cellular function in normal and disease states. The most intriguing feature of is that they can be isolated from cultured cells and be subcutaneously administered, making exosomes an exciting therapeutic delivery system. Indeed, exosome products are currently in Phase I and Phase II clinical trials for a variety of indications. Originally considered only a waste disposal system, they are now emerging as another class of signal mediators. Exosomes are secreted by any cell type and retrieved in every body fluid, such as blood, urine, saliva and amniotic liquid.

Recently, mesenchymal stem cells (MSCs) have been examined as a potential therapeutic for the treatment of a variety of human diseases, including cancer and immune disorders. MSCs are multipotent cells originating from the mesoderm of many tissues which have the capacity to self-renew and the ability to generate differentiated cells. In the last decade, MSCs have been extensively studies for their potential role in regenerative medicine, immunoregulation, neuroprotection, and anti-tumor effect. More recently, new interest has emerged regarding MSCs capacity to exert their therapeutic effect in a paracrine fashion by acting predominantly on the local environment. While efforts to use whole cell MSCs as therapy continue, a potentially more viable approach is to focus on one of the main vectors of these paracrine effects, exosomes.

Exosomes are relatively robust and stable secreted nanoparticles that contain many bioactive molecules and as such they appear attractive as gene and drug delivery vehicles. Exosomes have been shown to have immunomodulatory and regenerative properties. Because of these properties exosomes could be used for the treatment of numerous diseases and conditions in need innovative therapies such as cancer, diseases and disorders related to dysfunctions of the immune system, or transplant medicine.

### SUMMARY OF THE INVENTION

The present invention is based in part on the seminal discovery that extracellular vesicles (ECVs) derived from activated stromal cells have therapeutic properties. Specifically, ECVs derived from preactivated mesenchymal cells are effective in reducing cancer cell growth and metastasis as well as inducing tolerogenesis in immature dendritic cells. Additionally, ECVs derived from umbilical cord blood may be useful for immunosuppression and reduction of inflammation.

In one embodiment, the invention provides a pharmaceutical composition comprising an extracellular vesicle (ECV) produced by contacting a chorion stromal cell with an activator molecule and a pharmaceutically acceptable carrier. In one aspect, the activator molecule is a pro-inflammatory molecule, an anti-inflammatory molecule and/or an immune modulator. In certain aspects, the activator molecule is IFN-γ, Poly (I:C) or a combination thereof.

In another embodiment, the invention provides a pharmaceutical composition comprising an extracellular vesicle (ECV) isolated from umbilical cord blood plasma and a pharmaceutically acceptable carrier.

In an additional embodiment, the present invention provides a method of producing extracellular vesicles (ECVs) comprising contacting a stromal cell with an activator molecule, thereby producing extracellular vesicles (ECVs). In certain aspects, the stromal cell is a chorion stromal cell. In various aspects, the activator molecule is a pro-inflammatory molecule, an anti-inflammatory molecule and/or an immune regulating molecule. In one aspect, the ECV is an exosome.

In further embodiment, the invention provides an extracellular vesicle (ECV) produced by a method comprising contacting a stromal cell with an activator molecule, thereby producing extracellular vesicles (ECVs). In certain aspects, the stromal cell is a chorion stromal cell. In various aspects, the activator molecule is a pro-inflammatory molecule, an anti-inflammatory molecule and/or an immune modulator. In one aspect, the ECV is an exosome.

In one embodiment, the invention provides a method of reducing cancer cell proliferation comprising contacting a cancer cell with an extracellular vesicle (ECV), thereby reducing cancer cell proliferation. In an additional embodiment, the invention provides a method of reducing or inhibiting metastasis comprising contacting a cancer cell with an extracellular vesicle (ECV), thereby reducing or inhibiting metastasis. In a further embodiment, the invention provides a method of treating cancer comprising administering to a subject in need thereof an extracellular vesicle (ECV), thereby treating cancer. In various aspects, the ECV is produced by contacting a stromal cell with an activator molecule. In certain aspects, the activator molecule is a pro-inflammatory molecule, an anti-inflammatory molecule and/or an immune modulator. In various aspects, the activator molecule is IFN-γ, polyinosinic:polycytidylic acid (Poly (I:C)) or a combination of both IFN-γ and Poly (I:C). In one aspect, the stromal cell is a chorion stromal cell. In certain aspects, the cancer cell or cancer is ovarian cancer or pancreatic cancer.

In another embodiment, the invention provides method of inducing tolerogenesis in a cell comprising contacting the cell with an extracellular vesicle (ECV), thereby inducing tolerogenesis in the cell. In one aspect, the cell is an immature dendritic cell. In various aspects, the ECV is produced by contacting a stromal cell with an activator molecule. In certain aspects the activator molecule is a pro-inflammatory molecule, an anti-inflammatory molecule and/or an immune modulator. In various aspects, the activator molecule is IFN-γ, polyinosinic:polycytidylic acid (Poly (I:C)) or a combination of both IFN-γ and Poly (I:C). In one aspect, the stromal cell is a chorion stromal cell. In certain aspects, after tolerogenesis has been induced, the cell has reduced expression levels of CD209, CD83, CD1a, CD1c, HLA-DR or any combination thereof. In certain aspects, after tolerogenesis has been induced, the cell has increased expression levels of like PDL1, PDL2, CTLA-4, OX40L, CD85d or a combination thereof.

In an additional embodiment, the invention provides a method of reducing T cell lymphocyte proliferation comprising contacting a T cell lymphocyte with an extracellular vesicle (ECV), thereby reducing T cell lymphocyte proliferation. In a further embodiment, the invention provides a method of inducing immunosuppression and reducing inflammation comprising administering to a subject in need thereof an extracellular vesicle (ECV), thereby inducing immunosuppression and reducing inflammation. In various aspects, the ECV is isolated from umbilical cord blood plasma. In other aspects, the inflammation is related to frailty or aging.

### BRIEF DESCRIPTION OF THE DRAWINGS

FIG. 1 is a graphical representation of A2780 cell proliferation after 24, 48 and 72 hours of co-culture with MSCs. Ch-MSC: Chorion MSC; AM-MSC: Amniotic MSC; Lipo-MSC: Adipose MSC.

FIG. 2 is a graphical representation of A2780 cell proliferation after 24, 48 and 72 hours of co-culture with activated chorion MSCs.

FIG. 3 is a graphical representation of A2780 cell proliferation after 72 hours of co-culture with various activated MSCs.

FIGS. 4 A-B are visual representations of A2780 cells untreated and treated with exosomes 72 hr after treatment. A. Non-treated A2780 cells. B. A2780 cells treated with exosomes isolated from activated chorion MSCs.

FIG. 5 is a graphical representation of PANC2 cell proliferation after 1, 3 and 5 days of co-culture with activated chorion MSCs.

FIG. 6 is a graphical representation of A2780 cell proliferation after 24 and 48 hour of treatment with exosomes produced by activated chorion MSCs.

FIG. 7 is a schematic representation of the differentiation paths of immature dendritic cells into mature dendritic cells or tolerogenic dendritic cells.

FIG. 8 is a graphical representation of the percentage of tolerogenic and mature dendritic cells expressing CD14, CD209, CD83, CD85d, CD1a and HLADR cell surface markers after the treatment of immature dendritic cells with activated exosomes. maDC: mature dendritic cell; ToDC: tolerogenic dendritic cell.

FIGS. 9 A-B is a graphical representation of the expression of the cell surface proteins CD14, CD83, CD85d, CD1a and CD1c by control dendritic cells and dendritic cells treated with various activators. (A) MoDC: Dendritic cells derived from monocytes; Il-10: interleukin 10; Chorion: exosomes produced by chorion MSCs; Ch-INF-g: exosomes produced by chorion MSCs activated with interferon gamma; Ch-Pol: exosomes produced by chorion MSCs activated with Poly (I:C). (B) MoDC: Dendritic cells derived from monocytes; Ch-MSC: chorion MSCs; AMSC: amniotic MSCs; BM-MSC: bone marrow MSCs; IL10-DC: IL-10 dendritic cells; Villi-MSC; WJ-MSC: Wharton's Jelly MSCs; ASC: adipose stem cells.

FIG. 10 is a graphical representation of the percentage of CD83 positive cells obtained upon various treatment of immature dendritic cells. maDC: mature dendritic cells; IL10DC: interleukin 10; Ch-MSC-Poly: exosomes produced by chorion MSCs activated with Poly (I:C); Ch-MSC: exosomes produced by chorion MSCs; Ch-MSC-INFg: exosomes produced by chorion MSCs activated with interferon gamma.

FIGS. 11 A-B are graphical representations of the flow cytometry analysis of T cell proliferative state in the presence of CD3/CD28/CD2 activator and the presence or absence of exosomes. A. T cell proliferation in the absence of exosomes. B. T cell proliferation in the presence of exosomes.

FIG. 12 is a graphical representation of the flow cytometry analysis of T cell proliferative state, in the presence of CD3/CD28/CD2 activator, in response to exposure to various doses of exosomes. D4 act+PI: DAY 4 activated with CD3/CD28/CD3 tetramer and gated with propidium iodide for viability; D4 act UCBEXO1-2: DAY 4 activated with CD3/CD28/CD3 tetramer and gated with propidium iodide for viability in the presence of ½ the umbilical cord exosome dose; D4 act UCBEXO1-4: DAY 4 activated with CD3/CD28/CD3 tetramer and gated with propidium iodide for viability in the presence of ¼ the umbilical cord exosome dose please provide; D4 act UCBEXO1-8: DAY 4 activated with CD3/CD28/CD3 tetramer and gated with propidium iodide for viability in the presence of ⅛ the umbilical cord exosome dose.

FIG. 13 is a graphical representation of the effect of placental stromal cell exosomes on immune regulation. Left: percentage of CD4+ lymphocytes. Right: survival of NSG immunodeficient mice engrafted with human peripheral blood mono-nucleaded cells. PBMC: positive control, disease GvHD; Csa: negative control, cyclosporine A; Exo: exosomes from placental stromal cells.

FIGS. 14A-F are a graphical representation of the effect of umbilical cord plasma exosomes. A. Age adjustment based on methylation gene data compared to expected. B. Evaluation of IgG (left), IgA (middle) and IgM (right) levels before and after treatment. C. Evaluation of the level of lipoprotein A before and after treatment. D. Effect of the treatment on LH and FSH levels. E. Effect of the treatment on eGFR levels. F. effect of the treatment on Insulin Growth Factor 1 (IGF-1).

### DETAILED DESCRIPTION OF THE INVENTION

The present invention is based in part on the seminal discovery that extracellular vesicles (ECVs) derived from activated stromal cells have therapeutic properties. Specifically, ECVs derived from preactivated mesenchymal cells are effective in reducing cancer cell growth and metastasis as well as inducing tolerogenesis in immature dendritic cells. Additionally, ECVs derived from umbilical cord blood may be useful for immunosuppression and reduction of inflammation.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the invention provides a pharmaceutical composition comprising an extracellular vesicle (ECV) produced by contacting a chorion stromal cell with an activator molecule and a pharmaceutically acceptable carrier. In one aspect, the activator molecule is a pro-inflammatory molecule, an anti-inflammatory molecule and/or an immune modulator. In certain aspect, the activator molecule is IFN-γ, Poly (I:C) or a combination thereof.

In an additional embodiment, the invention provides a pharmaceutical composition comprising an extracellular vesicle (ECV) isolated from umbilical cord blood plasma, and a pharmaceutically acceptable carrier.

By “pharmaceutically acceptable” it is meant that the carrier, diluent or excipient must be compatible with the other ingredients of the formulation and not deleterious to the recipient thereof. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

In one embodiment, the present invention provides a method of producing extracellular vesicles (ECVs) comprising contacting a stromal cell with an activator molecule, thereby producing extracellular vesicles (ECVs). In one aspect, the stromal cell is a mesenchymal stem cell. In a specific aspect, the stromal cell is a chorion stromal cell. In an additional aspect, the activator molecule is a pro-inflammatory molecule, an anti-inflammatory molecule and/or an immune regulating molecule. In certain aspects, the activator is an interferon, a tumor necrosis factor, mTOR inhibitor, damage-associated molecular patterns (DAMP S)molecule, an/or pathogen associated molecular patterns (PAMPS) molecule. In specific aspects, the activator is INF-γ, polyinosinic:polycytidylic acid (Poly (I:C)) or a combination thereof. In various aspects, the ECV is an exosome.

As used herein, the terms “extracellular vesicle” and “ECV” are interchangeable and refer to exosomes, secreted cell-derived vesicles surrounded by a distinct phospholipid bilayer. Exosomes are a family of small nanoparticles having a diameter comprised between 30 and 100 nm. Exosomes are generated inside multivesicular endosomes or multivesicular bodies (MVBs) and are secreted when these compartments fuse with plasma membrane. Exosomes are enriched in endosome-derived components and also contain many bioactive molecules such as proteins, lipids, and nucleic acids including mRNAs, microRNAs (miRNAs), long noncoding RNAs (lncRNAs), transfer RNA (tRNA), genomic DNA, cDNA, and mitochondrial DNA (mtDNA). Exosomes may be released from multiple cell types, including reticulocytes, immunocytes, tumor cells, and MSCs. Exosome secretion is a general cellular function that plays an important role in the intercellular transfer of information, as such exosomes exert their function through paracrine effects. These secreted vesicles serve as cell-to-cell messengers that modify cellular function in normal state as well as in disease state. Exosomes resist degradation and contain membrane proteins potentially and are therefore useful for targeting and docking. The most intriguing feature of exosomes is that they can be isolated from cultured cells and be subcutaneously administered, making exosomes an exciting therapeutic delivery system. Indeed, exosome products are in Phase I and II clinical trials for a variety of indications. Originally considered only a waste disposal system, they are now emerging as another class of signal mediators. Exosomes are secreted by any cell type and retrieved in every body fluid, such as blood, urine, saliva and amniotic liquid. ECVs of the invention may be useful for their immunoregulation as well as anti-tumor effects. ECVs may be secreted by various different types of cells including stromal cells, mesenchymal stem cells and chorion stromal cells.

Stromal cells are connective tissue cells that support the function of the parenchymal cells of every organ of any organ. Fibroblasts and pericytes are among the most common types of stromal cells. Stromal cells can be derived from various tissues or organs, such as skin, heart, blood vessels, bone marrow, skeletal muscle, liver, pancreas, brain, foreskin or placenta.

Mesenchymal stem cells (MSCs) are multipotent cells originating from the mesoderm of many tissues, including bone marrow, liver, spleen, peripheral blood, adipose, placenta, and umbilical cord blood, and have the capacity to self-renew and the ability to generate differentiated cells such as osteoblasts, adipocytes and chondrocytes as well as myocytes and neurons. The terms mesenchymal stem cell (MSC) and marrow/mesenchymal stromal cell have been used interchangeably for, and encompasses, multipotent cells also derived from other non-marrow tissues, such as placenta, umbilical cord blood, adipose tissue, adult muscle, corneal stroma or the dental pulp of deciduous baby teeth. The cells do not have the capacity to reconstitute an entire organ. The youngest and most primitive MSCs can be obtained from umbilical cord tissue, namely Wharton's jelly and the umbilical cord blood. These MSCs may prove to be a useful source of MSCs for clinical applications due to their primitive properties.

In the last decade, MSCs have been extensively studied for their potential role in regenerative medicine, immunoregulation, neuroprotection, and anti-tumor effect. More recently, new interest has emerged regarding MSCs capacity to exert their therapeutic effect in a paracrine fashion by acting predominantly on the local environment. While efforts to use whole cell MSCs as therapy continue, a potentially more viable approach is to focus on one of the main vectors of these paracrine effects exosomes.

Chorionic membrane is one of the two fetal membranes associated with the developing fetus, along with the amnion. The chorion and the amnion together form the amniotic sac, that surrounds and protects the fetus. It is formed by extraembryonic mesoderm and the two layers of trophoblast that surround the embryo. Chorion stromal cells can be isolated from human chorionic membrane.

As used herein “activator molecule” refers to any molecule used to activate a stromal cell, i.e. to induce or enhance the production of exosomes by a stromal cell. The activator molecule of the invention might be a pro-inflammatory molecule, an anti-inflammatory molecule, an immune regulating molecule, chorionic villi, chorion trophoblast, syncytiotrophoblast, cytotrophoblast, interferons, tumor necrosis factors, interleukins, mTOR inhibitors, damage-associated molecular patterns (DAMPS) molecules, pathogen-associated molecular patterns (PAMPS) molecules, corticosteroids, calcineurin inhibitors and/or toll like receptor (TLR) activators. Specific examples of activator molecules include IFN-γ and Poly (I:C).

Inflammation is a protective response involving immune cells, blood vessels, and molecular mediators. The process of inflammation is initiated by resident immune cells, which upon activation release inflammatory molecules responsible for the clinical signs of inflammation. Resolution of inflammation may occur because of the short half-life of inflammatory molecules in vivo, or because of the production and release of anti-inflammatory molecules, the down regulation of pro-inflammatory molecules, the up-regulation of anti-inflammatory molecules, the apoptosis of pro-inflammatory cells, or the desensitization of receptors.

The phrase “pro-inflammatory molecule” refers to any molecule capable of inducing an inflammatory response. Examples of pro-inflammatory molecules include, but are not limited to, IL-1α, IL-1β, IL-12, IL-18, TNFα, IFNγ, GM-CSF, interferons, tumor necrosis factors, mTOR inhibitors, DAMPS and PAMPS.

Interferons (IFNs) are a group of signaling proteins made and released by host cells in response to the presence of several pathogens, such as viruses, bacteria, parasites, and also tumor cells. In a typical scenario, a virus-infected cell will release interferons causing nearby cells to heighten their anti-viral defenses. IFNs belong to the large class of proteins known as cytokines, molecules used for communication between cells to trigger the protective defenses of the immune system that help eradicate pathogens. Examples of IFNs include IFN-α, IFN-β, IFN-ε, IFN-κ and IFN-γ.

The tumor necrosis factor (TNF) superfamily refers to a superfamily of cytokines that can cause cell death. All TNF superfamily members form homotrimeric (or heterotrimeric in the case of LT-alpha/beta) complexes that are recognized by their specific receptors. Examples of TNF super family members include TNF, TNF-β, lymphotoxin-alpha, CD40L, CD27L, CD30L, FASL, 4-1BBL, OX40L and TRAIL.

mTOR inhibitors are a class of drugs that inhibit the mechanistic target of rapamycin (mTOR), which is a serine/threonine-specific protein kinase that belongs to the family of phosphatidylinositol-3 kinase (PI3K) related kinases (PIKKs). mTOR regulates cellular metabolism, growth, and proliferation by forming and signaling through two protein complexes, mTORC1 and mTORC2. The most established mTOR inhibitors are so-called rapalogs (rapamycin and its analogs). Examples of mTOR inhibitors include sirolimus, temsirolimus, ridaforolimus, ereolimus and PI3K inhibitors.

Damage-associated molecular patterns (DAMPs), also known as danger-associated molecular patterns, danger signals, and alarmin, are host biomolecules that can initiate and perpetuate a noninfectious inflammatory response. A subset of DAMPs are nuclear or cytosolic proteins. When released outside the cell or exposed on the surface of the cell following tissue injury, they move from a reducing to an oxidizing milieu, which results in their denaturation. Also, following necrosis, tumor DNA is released outside the nucleus, and outside the cell, and becomes a DAMP. Examples of DAMPS include HMGB1, DNA and RNA, S100 proteins, purine metabolites, monosaccharides and polysaccharides.

Pathogen-associated molecular patterns, or PAMPs, are molecules associated with groups of pathogens, that are recognized by cells of the innate immune system. These molecules can be referred to as small molecular motifs conserved within a class of microbes. They are recognized by toll-like receptors (TLRs) and other pattern recognition receptors (PRRs) in both plants and animals. A vast array of different types of molecules can serve as PAMPs, including glycans and glycoconjugates.

Toll-like receptors (TLRs) are a class of proteins that play a key role in the innate immune system. They are single, membrane-spanning, non-catalytic receptors usually expressed on sentinel cells such as macrophages and dendritic cells, that recognize structurally conserved molecules derived from microbes. Once these microbes have breached physical barriers such as the skin or intestinal tract mucosa, they are recognized by TLRs, which activate immune cell responses. The TLRs include TLR1, TLR2, TLR3, TLR4, TLR5, TLR6, TLR7, TLR8, TLR9, TLR10, TLR11, TLR12, and TLR13. Examples of TLR activators include polyinosinic:polycytidylic acid (Poly (I:C)) and lipopolysaccharide (LPS).

The phrase “anti-inflammatory molecule” refers to any molecule capable of inhibiting or reducing an inflammatory response. Examples of anti-inflammatory molecule include, but are not limited to, IL-1 receptor antagonist, IL-4, IL-6, IL-10, IL-11, IL-13, corticosteroids, calcineurin inhibitors, TGF-β, vitamin D and retinoic acid.

Corticosteroids are a class of steroid hormones that are produced in the adrenal cortex of vertebrates, as well as the synthetic analogues of these hormones. Two main classes of corticosteroids, glucocorticoids and mineralocorticoids, are involved in a wide range of physiologic processes, including stress response, immune response, and regulation of inflammation, carbohydrate metabolism, protein catabolism, blood electrolyte levels, and behavior. Some common naturally occurring steroid hormones are cortisol, corticosterone, and cortisone. Other examples of corticosteroids include prednisone, prednisolone, dexamethasone, budesonide, beclomethasone dipropionate, triamcinolone acetonide, fluticasone propionate, fluticasone furoate, flunisolide, methylprendisone and hydrocortisone.

Calcineurin inhibitors suppress the immune system by preventing interleukin-2 (IL-2) production in T cells. Examples of calcineurin inhibitors include cyclosporine and tacrolimus. Cyclosporine and tacrolimus bind to the intracellular immunophilins cyclophilin and FKBP-12, respectively. When bound, both molecules inhibit the phosphatase action of calcineurin, which is required for the movement of nuclear factors in activated T cells to the chromosomes where subsequent cytokine synthesis occurs. Decreased secretion of IL-2 prevents proliferation of the inflammatory response via B cells and T cells. The attenuated inflammatory response greatly reduces the overall function of the immune system.

The immune system is a system of biological structures and processes within an organism that protects against disease. This system is a diffuse, complex network of interacting cells, cell products, and cell-forming tissues that protects the body from pathogens and other foreign substances, destroys infected and malignant cells, and removes cellular debris: the system includes the thymus, spleen, lymph nodes and lymph tissue, stem cells, white blood cells, antibodies, and lymphokines.

The terms “immunoreactive cell”, “immune cells” or “immune effector cells” in the context of the present invention relate to a cell which exerts effector functions during an immune reaction. An “immunoreactive cell” preferably is capable of binding an antigen or a cell characterized by presentation of an antigen or an antigen peptide derived from an antigen and mediating an immune response. For example, such cells secrete cytokines and/or chemokines, secrete antibodies, recognize cancerous cells, and optionally eliminate such cells. For example, immunoreactive cells comprise T cells (cytotoxic T cells, helper T cells, tumor infiltrating T cells), B cells, natural killer cells, neutrophils, macrophages, and dendritic cells.

An immune disease or disorder is a dysfunction of the immune system. These disorders can be characterized in several different ways: by the component(s) of the immune system affected; by whether the immune system is overactive or underactive and by whether the condition is congenital or acquired. An immune disease or disorder is a dysfunction of the immune system. These disorders can be characterized in several different ways: by the component(s) of the immune system affected; by whether the immune system is overactive or underactive and by whether the condition is congenital or acquired. Immune diseases and disorders comprise autoimmune diseases or disorders, characterized by the dysfunction of the adaptive immune system, where adaptive immune B and T cells have lost their ability to differentiate self from non-self, and inflammatory diseases or disorders characterized by the dysfunction of the innate immune system, where innate immune cells inappropriately secrete inflammatory molecules such as cytokines. In the context of organ transplant self-antigen reactivity can induce damage to or destruction of the transplant, leading to its rejection. Deliberately induced immunosuppression is the main method used to prevent the body from rejecting the transplant. However immunosuppressive drugs have the potential to cause immunodeficiency.

As used herein, the terms “modulating an immune response”, “immunomodulation”, “immunoregulation”, and the like refer to either enhancing or inhibiting an immune response.

As such the terms “immunoregulator”, “immune regulating agent”, “immune modulator” and the like refer to any therapeutic agent that modulates the immune system. Examples of immune modulators include eicosanoids, cytokines, prostaglandins, interleukins, chemokines, checkpoint regulators, TNF superfamily members, TNF receptor superfamily members and interferons. Specific examples of immune modulators include PGI2, PGE2, PGF2, CCL14, CCL19, CCL20, CCL21, CCL25, CCL27, CXCL12, CXCL13, CXCL-8, CCL2, CCL3, CCL4, CCL5, CCL11, CXCL10, IL1, IL2, IL3, IL4, IL5, IL6, IL7, IL8, IL9, IL10, IL11, IL12, IL13, IL15, IL17, IL17, INF-α, INF-β, INF-ε, INF-γ, G-CSF, TNF-α, CTLA, CD20, PD1, PD1L1, PD1L2, ICOS, CD200, CD52, LTα, LTαβ, LIGHT, CD27L, 41BBL, FasL, Ox40L, April, TL1A, CD30L, TRAIL, RANKL, BAFF, TWEAK, CD40L, EDA1, EDA2, APP, NGF, TNFR1, TNFR2, LTβR, HVEM, CD27, 4-1BB, Fas, Ox40, AITR, DR3, CD30, TRAIL-R1, TRAIL-R2, TRAIL-R3, TRAIL-R4, RANK, BAFFR, TACT, BCMA, Fn14, CD40, EDAR XEDAR, DR6, DcR3, NGFR-p′75, and Taj. Other examples of immune modulators include tocilizumab (Actemra), CDP870 (Cimzia), enteracept (Enbrel), adalimumab (Humira), Kineret, abatacept (Orencia), infliximab (Remicade), rituzimab (Rituxan), golimumab (Simponi), Avonex, Rebif, ReciGen, Plegridy, Betaseron, Copaxone, Novatrone, natalizumab (Tysabri), fingolimod (Gilenya), teriflunomide (Aubagio), BG12, Tecfidera, and alemtuzumab (Campath, Lemtrada), prednisone, budesonide, prednisolone, calcineurin inhibitors, cyclosporine, tacrolimus, sirolimus, everolimus, zathioprine, leflunomide, mycophenolate, anakinra, certolizumab, etanercept, ixekizumab, secukinumab, tocilizumab, ustekinumab, vedolizumab, basiliximab, daclizumab, and muromonab.

The inflammasome is a multiprotein oligomer consisting of caspase 1, PYCARD, NALP and sometimes caspase 5. It is expressed in myeloid cells and is a component of the innate immune system. The exact composition of an inflammasome depends on the activator which initiates inflammasome assembly, e.g. dsRNA will trigger one inflammasome composition whereas asbestos will assemble a different variant. The inflammasome promotes the maturation of the inflammatory cytokines Interleukin 1β (IL-1β) and Interleukin 18 (IL-18). The inflammasome is responsible for activation of inflammatory processes. Because the pro-inflammatory pathway does not need Toll-like receptors (TLRs), inflammasomes can detect cytoplasmic DNA that may be threatening and strengthen their innate response. Inflammasomes have been shown to induce cell pyroptosis, a process of programmed cell death distinct from apoptosis. Inflammasome inducers are molecules that induce caspase-1 activation and IL-1β maturation by triggering the assembly of the NLRP3 or AIM2 inflammasome protein complexes. They act through various mechanisms, such as induction of cytosolic potassium efflux or phagosomal destabilization. Inflammasome inducers include Alum crystals, ATP, Chitosan ultrapure, Chitosan VacciGrade™, CPPD crystals, Hemozoin, MSU crystals, Nano-SiO2, Nigericin, TDB, Poly(dA:dT), Poly(dA:dT)/LyoVec™, Poly(dA:dT) rhodamine, FLA-PA ultrapure, FLA-ST, L18-MDP, MDP, Curdian, HKCAm Pustulan and Zymosan Depleted.

In another embodiment, the invention provides an extracellular vesicle (ECV) produced by the method comprising contacting a stromal cell with an activator molecule, thereby producing extracellular vesicles (ECVs). In certain aspects, the stromal cell is a mesenchymal stem cell. In a specific aspect, the stromal cell is a chorion stromal cell. In another aspect, the activator molecule is a pro-inflammatory molecule, an anti-inflammatory molecule and/or an immune modulator. In certain aspects, the activator molecule is IFN-γ, Poly (I:C) or a combination thereof. In various aspects, the ECV is an exosome. In one aspect, the cancer cell or cancer is ovarian cancer or pancreatic cancer.

In an additional embodiment, the invention provides a method of reducing cancer cell proliferation comprising contacting a cancer cell with an extracellular vesicle (ECV), thereby reducing cancer cell proliferation. In another embodiment, the invention provides a method of reducing or inhibiting metastasis comprising contacting a cancer cell with an extracellular vesicle (ECV), thereby reducing or inhibiting metastasis. In yet another embodiment, the invention provides a method of treating cancer comprising administering to a subject in need thereof an extracellular vesicle (ECV), thereby treating cancer. In certain aspects, the stromal cell is a mesenchymal stem cell. In a specific aspect, the stromal cell is a chorion stromal cell. In another aspect, the activator molecule is a pro-inflammatory molecule, an anti-inflammatory molecule and/or an immune modulator. In another aspect, the activator molecule is IFN-γ, Poly (I:C) or a combination thereof. In various aspects, the ECV is an exosome.

The term “cancer” refers to a group diseases characterized by abnormal and uncontrolled cell proliferation starting at one site (primary site) with the potential to invade and to spread to others sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof. “Metastasis” refers to the biologically process involved in the development of metastases. “Neoplasm” or “tumor” including grammatical variations thereof, means new and abnormal growth of tissue, which may be benign or cancerous. As such, the phrases “treating cancer” and “inhibiting metastasis” respectively refer to treating or inhibiting the development of primary tumor and to preventing, treating or inhibiting the development of metastases.

Exemplary cancers include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood′, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland′Cancer, Childhood; Sarcoma, Ewing's Family of Tumors; Sarcoma, Kaposi's; Sarcoma (OsteosarcomaVMalignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

“Cancer cell” or “tumor cell”, and grammatical equivalents refer to the total population of cells derived from a tumor or a pre-cancerous lesion, including both non-tumorigenic cells, which comprise the bulk of the tumor population, and tumorigenic stem cells (cancer stem cells).

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures). Treatment includes monotherapy approaches or combination therapy, for example, use of a ECVs alone or in combination with other therapeutic regimens. ECV-based therapy can be administered prior to, simultaneously with, or following other therapies, e.g., immunosuppressive therapy, chemotherapy, radiotherapy and the like.

The terms “administration of” and or “administering” should be understood to mean providing a therapeutic or pharmaceutical composition in a therapeutically effective amount to the subject in need of treatment. The terms are defined to include an act of providing an ECV composition of the invention to a subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration. The ECV compositions of the present invention may be processed in a number of way depending on the anticipated application and appropriate delivery or administration of the pharmaceutical composition. For example, the compositions may be formulated for injection.

The terms “therapeutically effective amount”, “effective dose,” “therapeutically effective dose”, “effective amount,” or the like refer to that amount of a therapeutic or pharmaceutical composition that will elicit the biological or medical response of a tissue, system, animal or human. Generally, the response is either amelioration of symptoms in a patient or a desired biological outcome.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

In some aspects several cancer treatments can be used in “combination therapy”, or “in combination”. The phrases “combination therapy”, “combined with” and the like refer to the use of more than one medication or treatment simultaneously. The ECVs of the present invention might for example be used in combination with other drugs or treatments to treat cancer. Specifically the administration of ECVs to a subject can be in combination with chemotherapy, radiation, or administration of a therapeutic antibody, for example. Such therapies can be administered prior to, simultaneously with, or following administration of ECVs. ECV-based therapy can be prior to, simultaneously with, or following other therapies, e.g., immunosuppressive therapy, chemotherapy, radiotherapy and the like. ECV-based therapy described herein can be prior to or following tumor resection, for example.

The term “chemotherapeutic agent” as used herein refers to any therapeutic agent having antineoplastic effect and used to treat cancer. In certain aspects, a chemotherapeutic agent, is a cytotoxic drug, an immunotherapeutic agent or radiation.

Examples of chemotherapeutic agents include, but are not limited to, Actinomycin, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fiuorouracil, Gemcitabine, Hydroxyurea, Idarubicin, Imatinib, lrinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, Vinorelbine, panitumamab, Erbitux (cetuximab), matuzumab, IMC-IIF 8, TheraCIM hR3, denosumab, Avastin (bevacizumab), Humira (adalimumab), Herceptin (trastuzumab), Remicade (infliximab), rituximab, Synagis (palivizumab), Mylotarg (gemtuzumab oxogamicin), Raptiva (efalizumab), Tysabri (natalizumab), Zenapax (dacliximab), NeutroSpec (Technetium (99mTc) fanolesomab), tocilizumab, ProstaScint (Indium-Ill labeled Capromab Pendetide), Bexxar (tositumomab), Zevalin (ibritumomab tiuxetan (IDEC-Y2B8) conjugated to yttrium 90), Xolair (omalizumab), MabThera (Rituximab), ReoPro (abciximab), MabCampath (alemtuzumab), Simulect (basiliximab), LeukoScan (sulesomab), CEA-Scan (arcitumomab), Verluma (nofetumomab), Panorex (Edrecolomab), alemtuzumab, CDP 870, natalizumab, Gilotrif (afatinib), Lynparza (olaparib), Perj eta (pertuzumab), Otdivo (nivolumab), Bosulif (bosutinib), Cabometyx (cabozantinib), Ogivri (trastuzumab-dkst), Sutent (sunitinib malate), Adcetris (brentuximab vedotin), Alecensa (alectinib), Calquence (acalabrutinib), Yescarta (ciloleucel), Verzenio (abemaciclib), Keytruda (pembrolizumab), Aliqopa (copanlisib), Nerlynx (neratinib), Imfinzi (durvalumab), Darzalex (daratumumab), Tecentriq (atezolizumab), Avelumab (Bavencio), Durvalumab (Imfinzi), Iplimumab (Yervoy) and Tarceva (erlotinib). Examples of immunotherapeutic agent include, but are not limited to, interleukins (Il-2, Il-7, Il-12), cytokines (Interferons, G-CSF, imiquimod), chemokines (CCL3, CCl26, CXCL7), immunomodulatory imide drugs (thalidomide and its analogues), MGA271, lirilumab, and BMS-986016.

In an additional embodiment, the invention provides a method of inducing tolerogenesis in a cell comprising contacting the cell with an extracellular vesicle (ECV), thereby inducing tolerogenesis in the cell. In one aspect, the cell is an immature dendritic cell. In an additional aspect, the ECV is produced by contacting a stromal cell with an activator molecule. In one aspect, the activator molecule is a pro-inflammatory molecule, an anti-inflammatory molecule and/or an immune modulator. In a specific aspect, the immune modulator is IFN-γ, Poly (I:C) or a combination thereof. In one aspect, the stromal cell is a chorion stromal cell. In a further aspect, after tolerogenesis has been induced, the cell has reduced expression levels of CD209, CD83, CD1a, CD1c, HLA-DR, or any combination thereof. In another aspect, after tolerogenesis has been induced, the cell has increased expression levels of PDL1, PDL2, CTLA-4, OX40L, CD85d or a combination thereof.

The term “tolerogenesis” refers to the induction of immunotolerance or immune acceptance, a state of unresponsiveness of the immune system to substances or tissue that have the capacity to elicit an immune response.

Dendritic cells (DC) are the most potent antigen-presenting cells of the immune system and are promising targets for immunotherapy having the objective to alleviate unwanted and excessive immune responses in allergic and autoimmune disorders. Indeed, tolerogenic DCs (a subtype of dendritic cells) exhibit numerous mechanisms to inhibit immune responses. Those tolerogenic DCs may be used for antigen-specific induction of tolerance in vivo, which would be beneficial for the therapy of allergic and autoimmune disease or in transplantation medicine.

Immature DC express low levels of surface proteins, collectively referred to as co-stimulation molecules (e.g., CD86, CD40, OX-40), produce little to no IL-12p70, and exhibit low nuclear factor kappa-light-chain-enhancer of activated B-cells (NF-κB) transactivational activity. Activation of DCs also results in CCR6 downregulation and CCR7 and CXCR4 upregulation. When antigens are acquired, DC undergo maturation that increase the expression and cell surface level of major histocompatibility complex (MHC) class II molecules for antigen presentation concurrent with the upregulation of co-stimulation molecules, and production of IL-12p70 to stimulate the division and activation of T-cells. Dendritic cells that acquire antigens but do not receive signals to undergo maturation maintain their immature state and can also present antigens to naïve T-cells in secondary lymphoid organs. In the absence of co-stimulation, these DC usually induce a state of anergy in target T-cells leading to peripheral tolerance.

The original concept of tolerance induction by DCs is attributed to low amounts of surface MHC and co-stimulatory molecules such as cluster of differentiation (CD) 80 and CD86. As such tolerogenic DCs are generally characterized by their minimal expression of co-stimulatory molecules CD80, CD83, CD86, MHC, and CCR7, and by the absence of secretion of inflammatory cytokines. Moreover, tolerogenic DCs released IL-10 and TGF-β which are both important for tolerance induction. In addition, several immunosuppressive features of tolerogenic DCs rely on induction of apoptosis in responding T cells including Fas cell surface death receptor (FasL/Fas) interactions, and tumor necrosis factor-related apoptosis-inducing ligand (TRAIL)/TRAILR engagement.

In one embodiment, the invention provides a method of reducing T cell lymphocyte proliferation comprising contacting a T cell lymphocyte with an extracellular vesicle (ECV), thereby reducing T cell lymphocyte proliferation. In one aspect, the ECV is isolated from umbilical cord blood plasma.

Immunoreactive cells comprise T cells or T lymphocytes, a type of lymphocyte that plays a central role in cell-mediated immunity. There are two major subtypes of T cells: the killer T cell (also referred to as CD8+ T cells) and the helper T cell (also referred to as CD4+ T cells). In addition, there are suppressor T cells which have a role in modulating immune response. Killer T cells only recognize antigens coupled to Class I MEW molecules, while helper T cells only recognize antigens coupled to Class II MHC molecules. These two mechanisms of antigen presentation reflect the different roles of the two types of T cell. A third minor subtype are the γδ T cells that recognize intact antigens that are not bound to MEW receptors.

T cell proliferation requires two independent signals. During an immune response, professional antigen-presenting cells (APCs) endocytose foreign material present antigen peptides to Class II MEW (first signal), allowing CD4+ T cells to activate the second signal involving an interaction between CD28 on the CD4+ T cell and the proteins CD80 (B7.1) or CD86 (B7.2) on the professional APCs. Once the two-signal activation is complete the T helper cell then allows itself to proliferate by releasing interleukin 2 (IL-2, a potent T cell growth factor called which acts upon itself in an autocrine fashion).

Umbilical cord blood plasma is the plasma isolated from the umbilical cord blood, which is the blood that remains in the placenta and in the attached umbilical cord after childbirth. The cord blood is composed of all the elements found in whole blood. It contains red blood cells, white blood cells, plasma, platelets and is also rich in hematopoietic stem cells. There are several methods for collecting cord blood, well known in the art.

In another embodiment, the invention provides a method of inducing immunosuppression and reducing inflammation comprising administering to a subject in need thereof an extracellular vesicle (ECV), thereby inducing immunosuppression and reducing inflammation. In one aspect, the ECV is isolated from umbilical cord blood plasma. In an additional aspect, the inflammation is related to frailty or aging.

The term “immunosuppression” refers to a reduction or an inhibition of the activation or efficacy of the immune system to induce an immune response. Some portions of the immune system have immunosuppressive effects and are involved into immune tolerance, however, immunosuppression can also be induced. Deliberately-induced immunosuppression is for example performed in the prevention of the rejection of a transplanted organ, or for the treatment of auto-immune diseases or disorders. Immunosuppression can also be non-deliberately induced, in in context of certain genetic diseases, in the context of cancer or due to certain chronic infections. Excessive immunosuppression can lead to immunodeficiency, a state in which the ability of the immune system is compromised or entirely absent.

The induction of immunosuppression is useful for treating subjects who have had an organ transplant and subjects with certain autoimmune disorders.

Organ transplantation is a medical procedure in which a damaged, missing non-functional organ is remove and replace by the same organ collected from a donor. When the recipient and the donor are two different subject, the transplantation is called an allograft. Organs that have been successfully transplanted include the heart, kidneys, liver, lungs, pancreas, intestine, and thymus. Tissues such as bones, tendons, cornea, skin, heart valves, nerves and veins can also be transplanted. Due to the genetic differences between the donor and the recipient, the main risk with allograft is the rejection of the transplant by the recipient's body. Indeed, the recipient's immune system identifies the transplanted organ as non-self and induces an immune response to eliminate it. Transplant rejection is one of the most challenging area in transplantation medicine, it requires to find the most appropriate donor-recipient match and to use immunosuppressant drugs. However, the use of immunosuppressive drug can cause immunodeficiency and thus can increase the susceptibility to opportunistic infections and decrease cancer immunosurveillance.

Auto-immune diseases or disorders that might require the use of immunosuppressant drug include, but is not limited to, rheumatoid arthritis, multiple sclerosis, myasthenia gravis, psoriasis, vitiligo, systemic lupus erythematosus, sarcoidosis, focal segmental glomerulosclerosis, Crohn's disease, Behcet's Disease, pemphigus, and ulcerative colitis.

“Immunosuppressive” or “immunosuppressant” drug refers to drug that inhibit or prevent the immune system to induce an immune response. Immunosuppressive drugs are used in immunosuppressive therapy, they include, but are not limited to, glucocorticoids, cytostatics, corticosteroids, calcineurin inhibitors, IMDH inhibitors, mTOR inhibitors, and antibodies.

Presented below are examples discussing of the described exosomes and uses of the exosomes. The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Evaluation of Ovarian Cancer Cell Proliferation after Co-Culture with Activated and Non-Activated MSCs

To evaluate the effect of MSCs on ovarian cancer cell proliferation, different types of MSCs (chorion-derived MSCs (Ch-MSC); amniotic-membrane-derived MSCs (AM-MSC); or adipose-tissue derived MSCs (Lipo-MSC)) were co-cultured with ovarian cancer cells A2780 plated on a porous membrane of trans-well insert to ensure that only paracrine effects will take effect. A2780 cells plated in tissue culture plates served as a control. The cells were co-cultured for 72 hours with cell number counts taken 24 hours, 48 hours and 72 hours after the initiation of the co-culture.

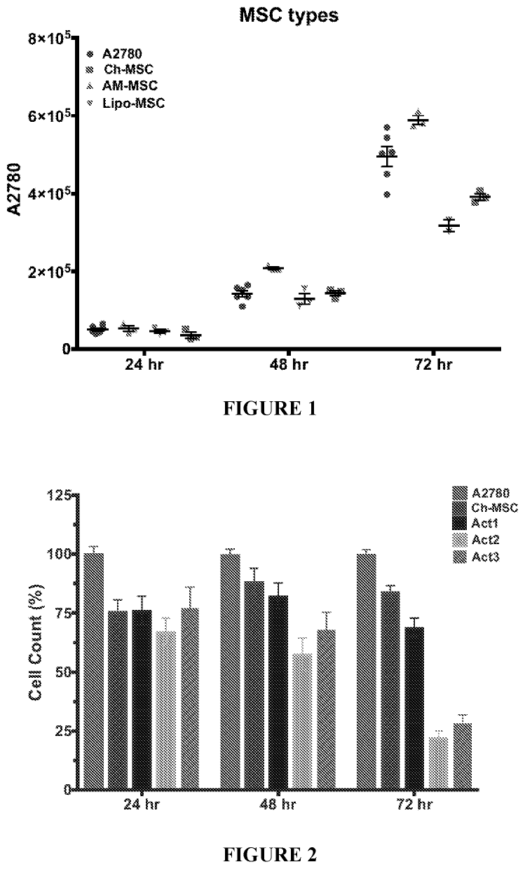

As shown in FIG. 1, the proliferation of ovarian cancer cells was reduced when co-cultured with MSCs. However, some MSCs were better at inhibiting proliferation than other types of MSCs. Specifically, chorion MSCs displayed a better capability at inhibiting proliferation of ovarian cancer cells.

To further assess the effect on ovarian cancer cell proliferation, chorion MSCs were activated with IFN-γ, Poly (I:C) and a combination of both molecules for 24 hours to simulate inflammation. The cells were then washed twice and cultured without activators for 24 hours. Afterward, the chorion MSCs were co-cultured with A2780 cells. A2780 cells co-cultured with themselves or with non-activated chorion MSCs (Ch-MSC) served as controls. The MSCs were co-cultured with A2780 cells for 72 hours with cell number counts taken 24 hour, 48 hours and 72 hours after the initiation of the co-culture.

As shown in FIG. 2, the inhibition of A2780 cell proliferation by the non-activated and activated Ch-MSCs was confirmed as early as 24 hour after initiation of co-culture. IFN-γ was more efficient at inhibiting the proliferation of ovarian cancer cells when used to activate chorion MSCs. This effect was confirmed at both 48 and 72 hour time-points.

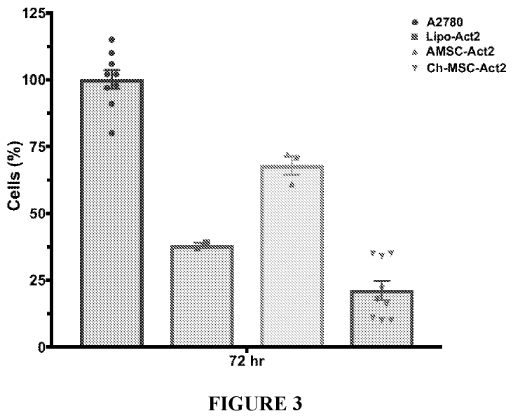

IFN-γ was then used to activate adipose-tissue derived MSCs (Lipo-Act2), amniotic-membrane-derived MSCs (AMSC-Act2), and chorion-derived MSCs (Ch-MSC-Act2) (FIG. 3) to assess their effect on ovarian cell proliferation. The MSCs were co-cultured with the A2870 cells for 72 hrs. Proliferation of the ovarian cancer cells were evaluated by counting the number of cells.

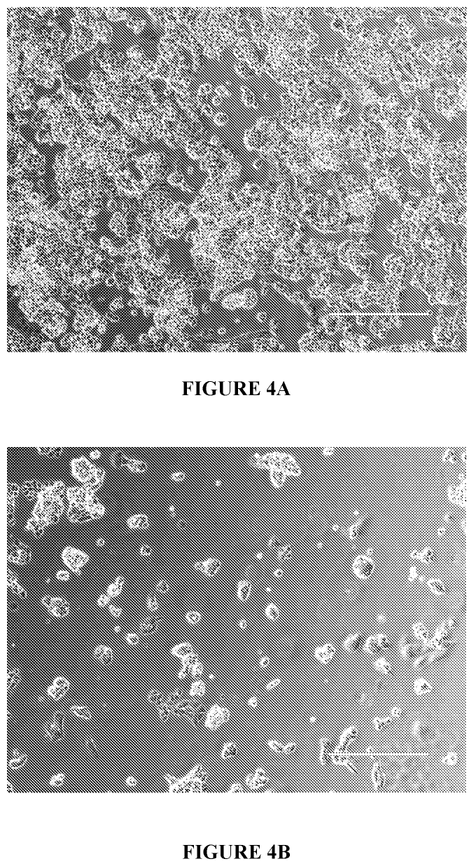

As shown in FIG. 3, activated chorion MSCs were found to be better at inhibiting proliferation of ovarian cancer cells as compared to adipose-tissue derived MSCs and amniotic-membrane-derived MSCs, when activated by IFN-γ. Indeed, IFN-γ-activated chorion MSCs were found capable of reducing ovarian cancer cell proliferation by 80% (see FIGS. 4 A-B).

### Example 2

### Evaluation of Pancreatic Cancer Cell Proliferation after Co-Culture with Activated MSCs

To assess the effect of activated MSCs on pancreatic cancer cell proliferation, chorion MSCs were activated with IFN-γ, Poly (I:C), and INF-γ+ Poly (I:C) for 24 hours, then washed twice and culture without activators for 24 hours. The chorion MSCs were then co-cultured with pancreatic cancer cells. Pancreatic cancer cells were plated on the porous membrane of transwell insert and then co-cultured with the activated chorion MSCs for 5 days. Pancreatic cancer cells co-cultured with pancreatic cancer cells or with non-activated chorion MSCs (Ch-MSC) served as controls. Cell number counts were taken at day 1, day 3 and day 5 after the initiation of the co-culture.

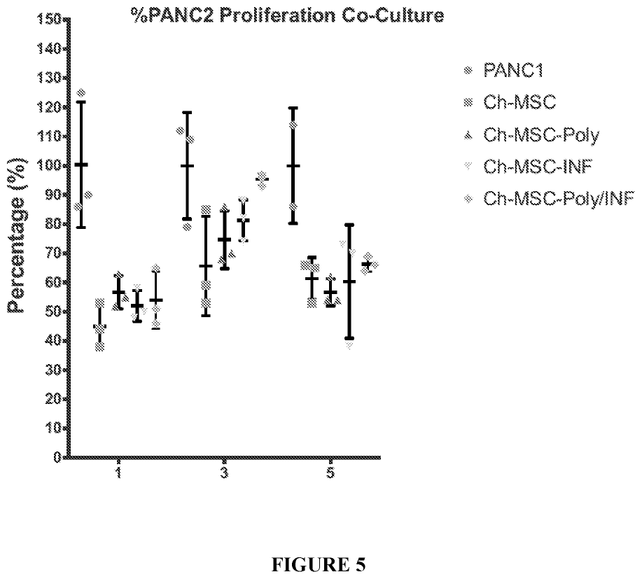

As shown in FIG. 5, the inhibitory effect of activated Ch-MSCs on PANC2 proliferation was established as early as day 1 following initiation of co-culture. The data show that pre-activation of chorion MSCs enhances the ability of chorion MSCs to inhibit the proliferation of pancreatic cancer cells.

### Example 3

### Evaluation of Ovarian Cancer Cell Proliferation after Treatment with Exosomes Produced by Activated MSCs

The effect of exosomes isolated from activated MSCs on ovarian cancer cell proliferation was examined. Chorion MSCs were activated with IFN-γ or Poly (I:C), as described in Example 1. Cell-free exosomes were isolated from chorion MSCs and quantified. Exosomes were isolated from conditioned media using centrifugation and ultracentrifugation process, then quantified using a colometric assay. A2780 cells were treated with bug of exosomes isolated from non-activated chorion MSCs (10 ug+Ch-MSC-Exo), 10 ug of exosomes isolated from chorion MSCs activated with IFN-γ (10 ug+Ch-MSC-Act1-Exo), or 10 ug of exosomes isolated from chorion MSCs activated with Poly (I:C) (10 ug+Ch-MSC-Act2-Exo). Un-activated chorion MSCs and untreated A2780 cells served as controls. The cells were treated once with the exosomes or controls for 48 hours with cell number counts taken at 24 hours and 48 hours after the initiation of the treatment.

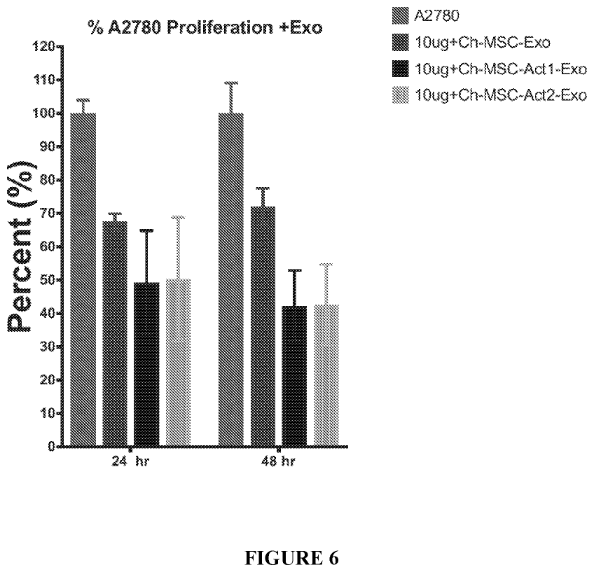

As shown in FIG. 6, exosomes produced by activated chorion MSCs inhibited proliferation of the A2780 cells. Indeed, it was shown that cell-free isolated exosomes were capable of reducing of ovarian cancer cell proliferation by 60%.

### Example 4

### Analysis of Exosome Anti-Proliferative Components

Individual batches of exosomes isolated from activated MSC as described above will then be lysed and subjected to microarray assays to identify those components of the exosome responsible for the observed anti-proliferative effects. Following the identification of such a candidate(s), mesenchymal cell line(s) that overexpresses the identified exosome constituents will be created and activated as described above. These cell lines will then be used to produce a virtually limitless supply of cancer-inhibiting exosomes in vitro that can be used as a commercially viable therapeutic. The exosomes will then be used in in vitro and in vivo toxicity and efficacy testing in mouse models of ovarian cancer, including the determination of the IC50. The exosomes produced will be a powerful adjunctive/adjuvant treatment for ovarian cancer enveloped within an innovative biological drug delivery device.

### Example 5

### Evaluation of Tolerogenesis Induction by Exosomes Produced by Activated MSCs

It has been shown that injection of MSCs can improve tolerogenesis in clinical settings (Zhoa et al. (2016); J. Cell. Immunotherapy 2(1):3-20). However, the exact mechanism of this effect has not been thoroughly explored and the compounds causing the effect have not been identified. It has been shown that exosomes are responsible for these tolerogenic effects mediated by MSCs. Six types of MSCs have been cultured as previously described, and activated with different activators and combination of activators.

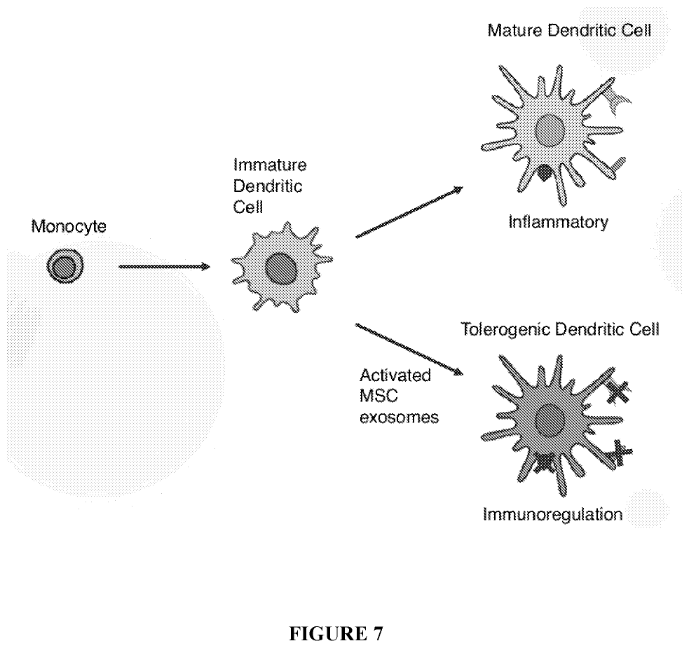

To assess tolerogenic effect of exosomes isolated from activated MSCs, monocytes were differentiated to dendritic cells using inflammatory stimuli (LPS), then were treated with exosomes isolated from MSCs activated with Poly (I:C) or IFN-γ and the expression of dendritic cell surface markers were evaluated (see FIG. 7).

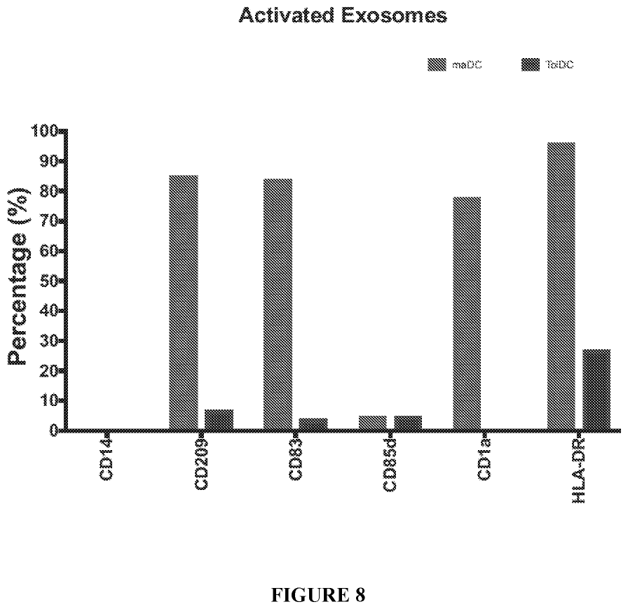

As shown in FIG. 8, dendritic cell the expression level of surface markers CD209, CD83, CD85d, CD1a, and HLA-DR was determined after exposure to the exosomes. Activated chorion MSCs produced exosomes capable of inducing tolerogenesis, as observed by the percentage of tolerogenic dendritic cells. Therefore, activated chorion MSC produced exosomes that can substantially exert tolerogenic effects in vitro through a paracrine mechanism.

To further characterize the tolerogenic effect of chorion MSCs derived exosomes, monocyte-immature dendritic cells were exposed to exosomes isolated from chorion MSCs activated by IL-10, INF-γ and Poly (I:C) and the expression of the cell surface protein CD14, CD83, CD85d, CD1a and CD1c was evaluated after an inflammatory molecule, Lipossacharide from Escherichia Coli (LPS), was introduced. Immature dendritic cells exposed to LPS and no exosomes served as controls.

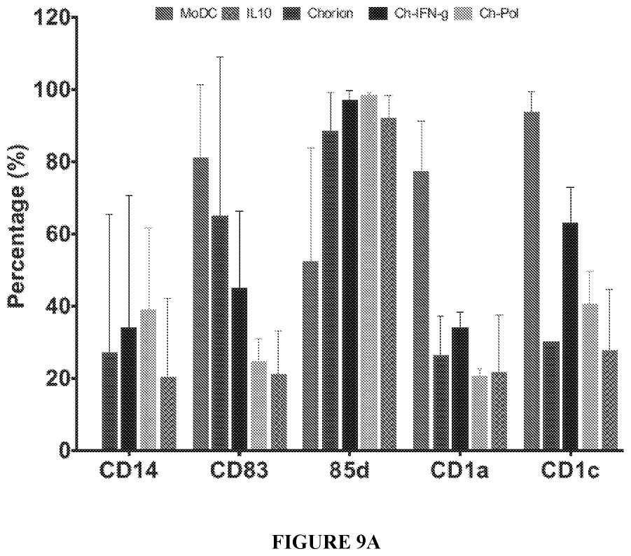

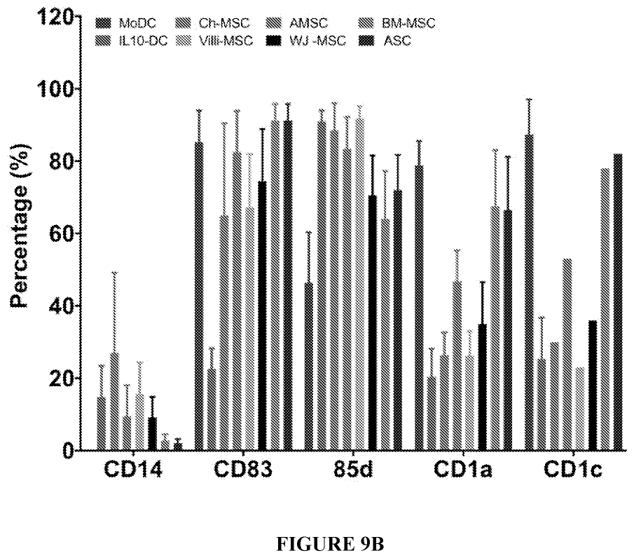

As shown in FIG. 9A, tolerogenic dendritic cells obtained after treatment with either exosomes isolated from chorion MSC activated with Poly (I:C) or with INF-γ, presented high levels of CD85d, CD14 and lower level of expression of CD83, CD1a and CD1c. Therefore, activated chorion MSCs were highly potent to generate robust tolerogenic dendritic cells. As shown in FIG. 9B, chorion stromal cells, and cells from the chorion plate (villi, syncytiotrophoblast, cytotrophoblast and trophoblast) also promote tolerogenesis.

Additionally, a dose-response experiment was performed. Immature dendritic cells were exposed to several doses of exosomes isolated from chorion MSCs activated with Poly (I:C) (Ch-MSC-Poly 1 ug, Ch-MSC-Poly 5 ug, and Ch-MSC-Poly 10 ug) or IFN-γ (Ch-INF-g 1 ug, Ch-INF-g 5 ug, and Ch-INF-g 01 ug). Immature dendritic cells exposed to interleukin 10 and exosomes isolated from non-activated chorion MSCs as well as mature dendritic cells served as controls. The percentage of CD83+ mature dendritic cells was then evaluated.

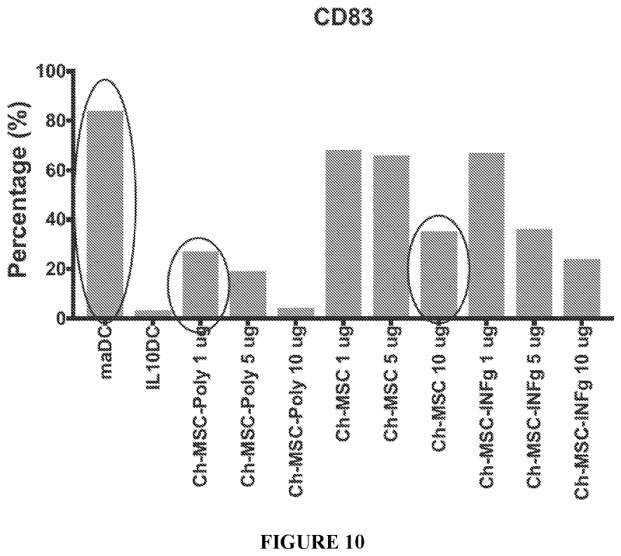

As shown in FIG. 10, the highest percentage of expression of CD83 was found in mature dendritic cells and IL10 treatment was highly efficient at reducing expression of CD83. Exosomes isolated from chorion MSCs were found to generate a moderate reduction of CD83 expression, even at high dose (e.g. 10 ug). Exosomes isolated from Poly (I:C)-activated chorion MSCs were found to strongly inhibit the expression of CD83 demonstrating the induction of tolerogenic differentiation of the immature dendritic cell.

### Example 6

### Analysis of Exomsome Tolerogenic Components

Individual batches of exosomes isolated from activated MSC as described above could then be lysed and used in microarray assays to identify those components of the exosome responsible for the observed tolerogenic effect. Following the identification of such a candidate, a mesenchymal cell line that overexpresses the identified exosome constituents will be created and activated as described above. Those MSCs would then be used to produce a virtually limitless supply of tolerogenic inducing exosomes in vitro that can be used as a commercially viable therapeutic. The exosomes will then be used in in vitro and in vivo toxicity and efficacy testing in mouse models of tolerogenesis. Additionally, the best therapeutic dose for the identified component will be determined. Further, the component exhibiting the primary tolerogenesis effect will be isolated. This innovative approach could reduce or replace the use of immunosuppressants and avoid risks, chronic side effect, and costs associated with them. Because there are no drugs on the current market specifically targeted to promote tolerogenesis in the body, the innovative approach outlined in this proposal would fill a much-needed gap in the field and provide an alternative to broad spectrum immunosuppressants. Especially this innovative procedure could lead to forward progress in the treatment of Graft vs Host Disease, post-transplantation.

### Example 7

### Evaluation of T Cell Proliferation Modulation by Exosomes Isolated from Blood Cord Plasma

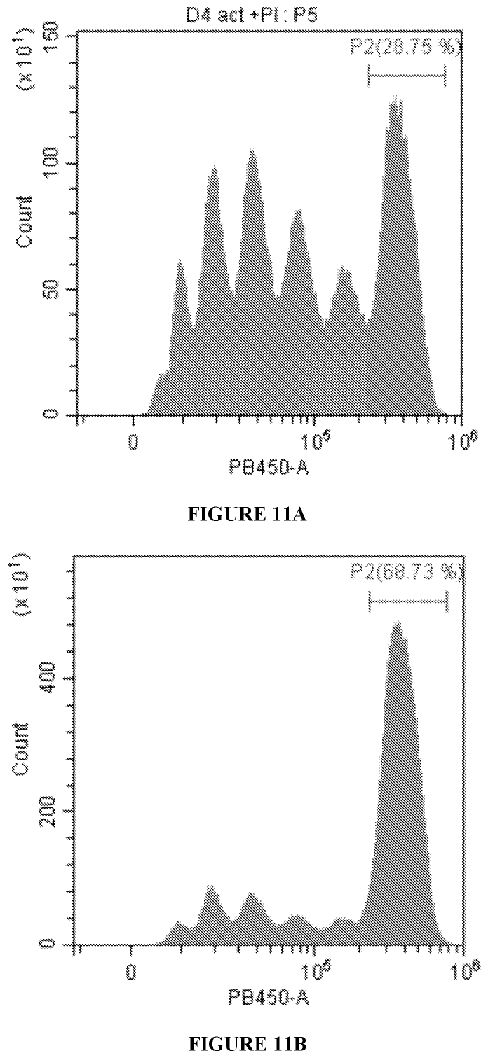

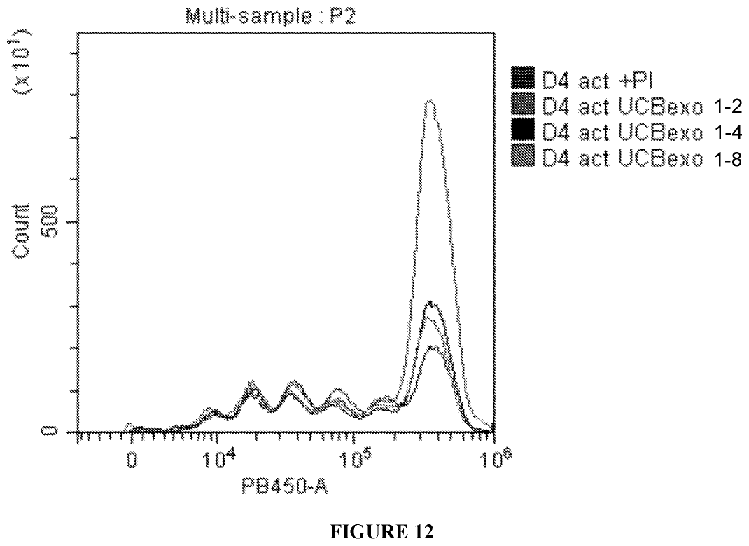

To determine whether exosomes could be used to induce immunosuppression and to reduce inflammation, exosomes were isolated from umbilical cord blood plasma and cultured with T cell lymphocytes. Exosomes were isolated from plasma using filtration, centrifugation and ultracentrifugation process, then quantified using a colometric assay. The activation of T cells from a non proliferative stage (Phase 0) to a proliferative phase was evaluated by flow cytometry. T cells were activated in the presence of CD3/CD28/CD2, treated with exosomes isolated from umbilical cord blood plasma, stained with propidium iodine, and analyzed by flow cytometry to evaluate their proliferative state. T cells not treated with exosomes served as control. As shown in FIG. 10A, activated T cells not treated with exosomes entered in a proliferative phase, as shown by the presence of only 29% of the cells in the non-proliferative phase. However, as shown in FIG. 10B, T cells treated with exosomes isolated from umbilical cord blood plasma were mostly in the non-proliferative phase with 69% of the T cells in the non-proliferative phase. Moreover, as shown in FIG. 11, the inhibition of the entry of T cells into a proliferative phase is dose dependent.

Exosomes isolated from umbilical cord blood plasma were found efficient to reduce the proliferation of T cells upon activation, making them an ideal candidate for immunosuppression and reduce the inflammation related to frailty and aging.

### Example 8

### Evaluation of Exosomes Isolated from Cord Blood

Extracellular vesicles from human umbilical cord plasma, due to its anti-inflammatory nature are likely to have anti-aging and anti-frailty properties when concentrated and applied regularly. The antiaging and anti-frailty effect of concentrated EVs from umbilical cord blood plasma will be tested in a 20-patient clinical trial in which DNA methylation, telomere length and other pertinent physical evaluation will be performed (hair color, balance, grip strength etc.). Low volume(concentrate), repeat dosage by subQ/IM injections will be used.

Although the invention has been described with reference to the above examples, it will be understood that modifications and variations are encompassed within the spirit and scope of the invention. Accordingly, the invention is limited only by the following claims.

### Example 9

### Evaluation of the Effect of Placental Stromal Cells Exosomes on Immunoregulation

Exosomes isolated from placental stromal cells were evaluated for their impact on immune-regulation, as compared to an known immunosuppressant.

A humanized mice model of graft versus host disease (GVHD), obtained by using NSG immunodeficient mice from Jackson laboratories engrafted with human peripheral blood mono-nucleated cells was used. The mice were either non-treated and considered as a positive control (PBMC: positive disease GVHD), treated with Cyclosporin A, which is a known immunosuppressant, and considered as a negative control (CsA), or treated with selected exosomes collected from placental stromal cells (Exo).

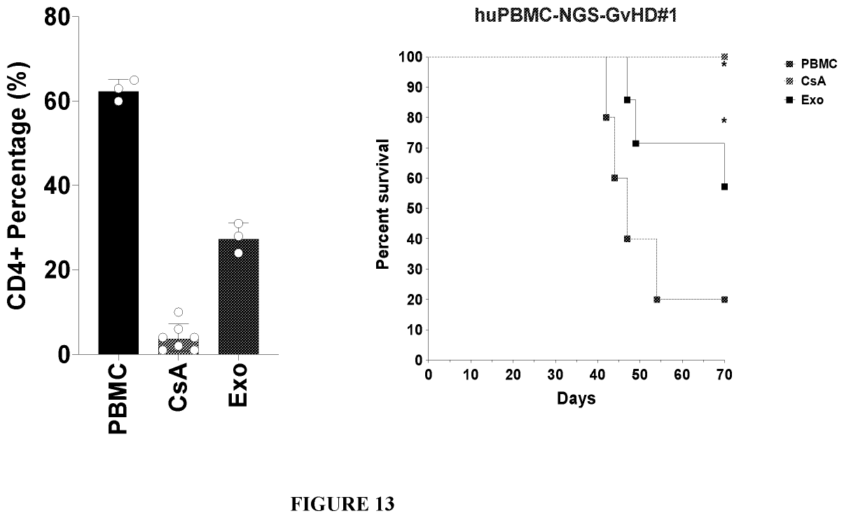

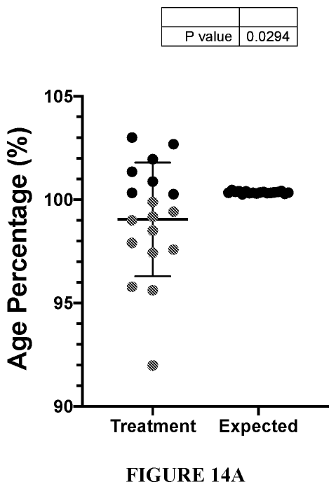

As illustrated in FIG. 13, PBMC non-treated mice presented a high percentage (60%) of CD4+ lymphocytes, which was associated with a high mortality rate (around 20% of the mice survived after 70 days). Mice treated with cyclosporine A presented low CD4+ lymphocytes percentages and a 100% survival rate at the end of the study. Mice treated with exosomes collected from placental stromal cells presented a reduction of CD4+ lymphocytes and an increased survival in vivo, describing how exosomes were capable inducing immune-regulation, as shown by the significant reduction of CD4+ cells and the increase in survival.

### Example 10

### Evaluation of the Effect of Umbilical Cord Plasma Exosome

Exosomes isolated from umbilical cord plasma were evaluated for their impact on various disease risk in a human trial including a healthy population with patients aged between 65 and 96.

The healthy volunteers were treated with exosomes isolated from umbilical cord plasma, and several parameters were evaluated prior to and after the treatment.

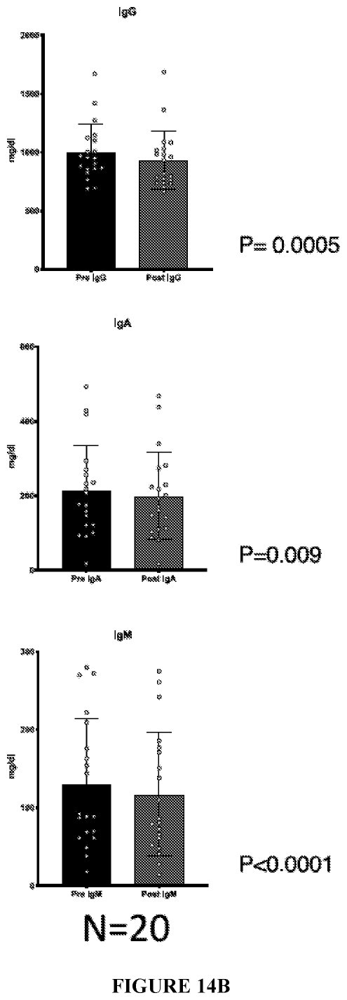

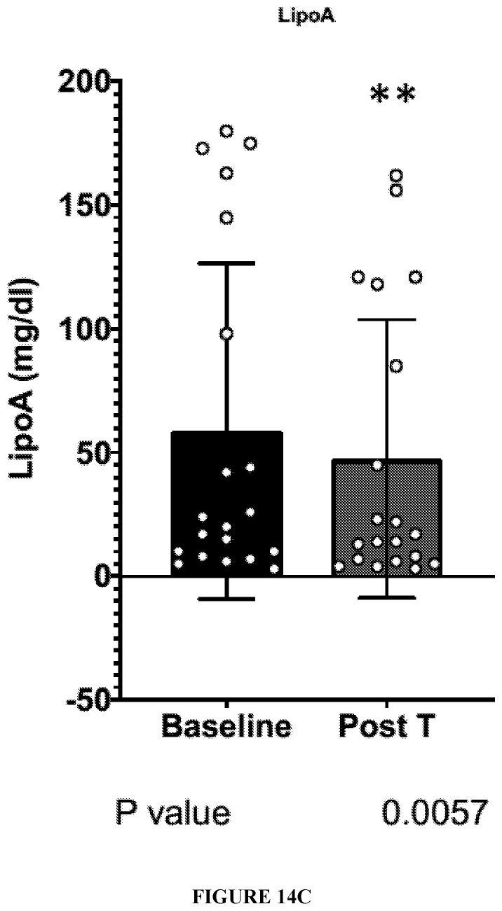

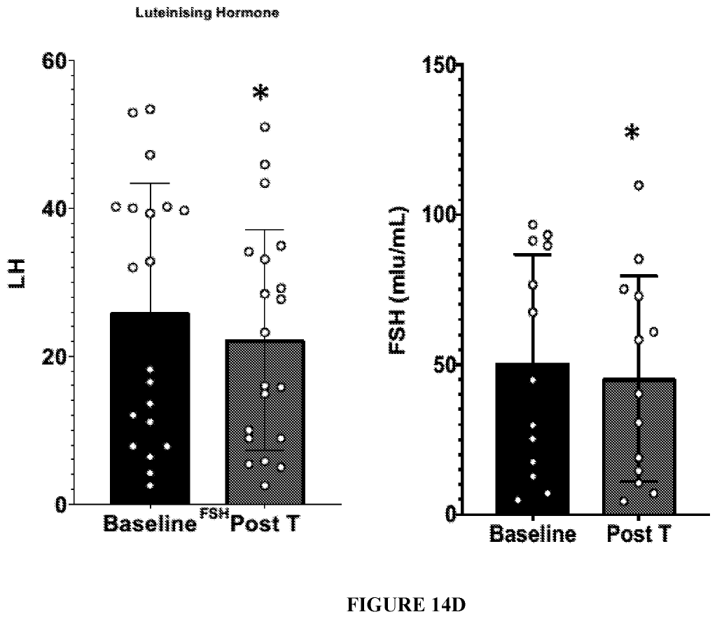

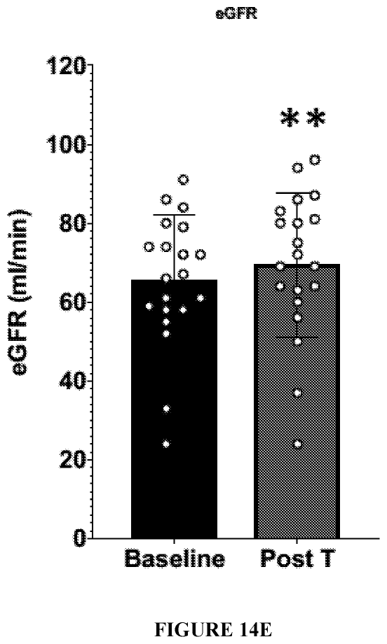

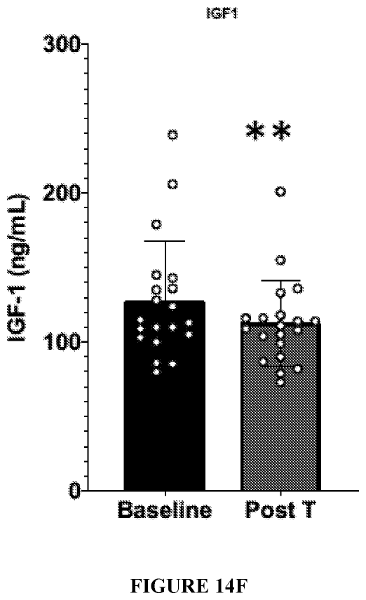

Based on methylation gene data, the age of the volunteers was evaluated after treatment with the umbilical cord plasma exosomes. As illustrated in FIG. 14A, as compared to the real age, the umbilical cord plasma exosomes “reduced” the ages of the volunteers.

The levels of immunoglobulins G, A, and M, which are related to autoimmunity, were evaluated before and after treatment. As illustrated in FIG. 14B, IgG, IgA and IgM levels were all reduced, suggesting a role for umbilical cord plasma exosomes in immune regulation and autoimmunity.

Lipoproteins levels, and especially lipoprotein A level, which is used to classify people at risk of heart disease, were evaluated before and after treatment. As illustrated in FIG. 14C, LipoA levels were decreased after treatment, which moved he average of the group go from “very high risk” of heart disease” to safer “high risk” group. This suggest a protective role for umbilical cord plasma exosomes against heart disease.

The levels of LH and FSH hormones, which are associated with cancer risk in elder populations, were evaluated before and after treatment. As illustrated in FIG. 14D, both LH and FSH levels were lowered by the treatment, suggesting a protective role for umbilical cord plasma exosomes against cancer.

The level of eGFR, which is representative of kidney function, was evaluated before and after treatment. As illustrated in FIG. 14E, eGFR level was increased by the treatment, suggesting a role for umbilical cord plasma exosomes on kidney function.

The levels of Insulin Growth Factor 1 (IGF-1), which is associated with cancer risk in elder populations, was evaluated before and after treatment. As illustrated in FIG. 14F, IGF-1 level was lowered by the treatment, suggesting a protective role for umbilical cord plasma exosomes against cancer.

What is claimed is:

1. A pharmaceutical composition comprising an extracellular vesicle (ECV) produced by contacting a chorion stromal cell with an activator molecule and a pharmaceutically acceptable carrier.

2. The pharmaceutical composition of claim 1, wherein the activator molecule is a pro-inflammatory molecule, an anti-inflammatory molecule and/or an immune modulator.

3. The pharmaceutical composition of claim 1, wherein the activator molecule is IFN-γ, Poly (I:C) or a combination thereof.

4. A pharmaceutical composition comprising an extracellular vesicle (ECV) isolated from umbilical cord blood plasma, and a pharmaceutically acceptable carrier.

5. A method of producing extracellular vesicles (ECVs) comprising contacting a stromal cell with an activator molecule, thereby producing extracellular vesicles (ECVs).

6. The method of claim 5, wherein the stromal cell is a chorion stromal cell.

7. The method of claim 5, wherein the activator molecule is a pro-inflammatory molecule, an anti-inflammatory molecule and/or an immune modulator.

8. The method of claim 7, wherein the pro-inflammatory molecule is selected from the group consisting of IL-1α, IL-1β, IL-12, IL-18, TNFα, IFNγ, a mTOR inhibitor, a DAMPS molecule, a PAMPS molecule, LPS, Poly (I:C) Poly (I:C) and GM-CSF.

9. The method of claim 7, wherein the anti-inflammatory molecule is selected from the group consisting of IL-1 receptor antagonist, IL-4, IL-6, IL-10, IL-11, and IL-13.

10. The method of claim 7, wherein the immune modulator is selected from the group consisting of corticosteroids, prednisone, budesonide, prednisolone, calcineurin inhibitors, cyclosporine, tacrolimus, mTOR inhibitors, sirolimus, everolimus, IMDH inhibitors, zathioprine, leflunomide, mycophenolate, abatacept, adalimumab, anakinra, certolizumab, etanercept, golimumab, infliximab, ixekizumab, natalizumab, rituximab, secukinumab, tocilizumab, ustekinumab, vedolizumab, monoclonal antibodies, basiliximab, daclizumab, and muromonab.

11. The method of claim 5, wherein the ECV is an exosome.

12. An extracellular vesicle (ECV) produced by the method of claim 5.

13. A method of reducing cancer cell proliferation comprising contacting a cancer cell with an extracellular vesicle (ECV), thereby reducing cancer cell proliferation.

14. A method of reducing or inhibiting metastasis comprising contacting a cancer cell with an extracellular vesicle (ECV), thereby reducing or inhibiting metastasis.

15. A method of treating cancer comprising administering to a subject in need thereof an extracellular vesicle (ECV), thereby treating cancer.

16. The method of any of claim 13-15, wherein the ECV is produced by contacting a stromal cell with an activator molecule.

17. The method of claim 16, wherein the activator molecule is a pro-inflammatory molecule, an anti-inflammatory molecule and/or an immune modulator.

18. The method of claim 16, wherein the activator molecule is IFN-γ, Poly (I:C) or a combination of IFN-γ and Poly (I:C).

19. The method of claim 16, wherein the stromal cell is a chorion stromal cell.

20. The method of any of claim 13-15, wherein the cancer cell or cancer is ovarian cancer or pancreatic cancer.

21. A method of inducing tolerogenesis in a cell comprising contacting the cell with an extracellular vesicle (ECV), thereby inducing tolerogenesis in the cell.

22. The method of claim 21, wherein the cell is an immature dendritic cell.

23. The method of claim 21, wherein the ECV is produced by contacting a stromal cell with an activator molecule.

24. The method of claim 23, wherein the activator molecule is a pro-inflammatory molecule, an anti-inflammatory molecule and/or an immune modulator.

25. The method of claim 23, wherein the activator molecule is IFN-γ, Poly (I:C) or a combination of IFN-γ and Poly (I:C).

26. The method of claim 23, wherein the stromal cell is a chorion stromal cell.

27. The method of claim 21, wherein, after tolerogenesis has been induced, the cell has reduced expression levels of CD209, CD83, CD1a, CD1c, HLA-DR, or any combination thereof.

28. The method of claim 21, wherein, after tolerogenesis has been induced, the cell has increased expression levels of PDL1, PDL2, CTLA-4, OX40L, CD85d or a combination thereof.

29. A method of reducing T cell lymphocyte proliferation comprising contacting a T cell lymphocyte with an extracellular vesicle (ECV), thereby reducing T cell lymphocyte proliferation.

30. A method of inducing immunosuppression and reducing inflammation comprising administering to a subject in need thereof an extracellular vesicle (ECV), thereby inducing immunosuppression and reducing inflammation.

31. The method of claim 29 or 30, wherein the ECV is isolated from umbilical cord blood plasma.

32. The method of claim 29 or 30, wherein the inflammation is related to frailty or aging.
